(19) United States
(12) Patent Application Publication
(10) Pub. No.: US 2025/0169679 A1
(43) Pub. Date: May 29, 2025

(54) MEDICAL DEVICE CAPS AND RELATED SYSTEMS AND METHODS OF USE

(71) Applicant: Boston Scientific Scimed, Inc., Maple Grove, MN (US)

(72) Inventors: Kapil GUPTA, Boston, MA (US); Ryan V. WALES, Northborough, MA (US)

(73) Assignee: Boston Scientific Scimed, Inc., Maple Grove, MN (US)

(21) Appl. No.: 18/958,443

(22) Filed: Nov. 25, 2024

Related U.S. Application Data
(60) Provisional application No. 63/603,175, filed on Nov. 28, 2023.

(51) Int. Cl. A61B 1/00; A61B 1/12

(52) U.S. Cl. CPC A61B 1/00137; A61B 1/00091; A61B 1/00101; A61B 1/126

(57) ABSTRACT

In some aspects, the techniques described herein relate to a cap for a medical device, including: a cylindrical body including a central lumen extending longitudinally through the cylindrical body, wherein a radially-inward facing surface of the cylindrical body extends around the central lumen; and a first protruding portion extending from the radially-inward facing surface, wherein the first protruding portion is configured to redirect fluid supplied from a fluid lumen of the medical device.

### CROSS-REFERENCE TO RELATED APPLICATIONS

This application claims the benefit of priority of U.S. Provisional Patent Application No. 63/603,175, filed on Nov. 28, 2023, the entirety of which is incorporated herein by reference.

### TECHNICAL FIELD

Various aspects of this disclosure relate generally to caps for medical devices, and related systems and methods. In particular, aspects of this disclosure relate to caps for endoscopes or other medical devices, among other aspects.

### BACKGROUND

Endoscopes have attained great acceptance within the medical community since they provide a means for performing procedures with minimal patient trauma, while enabling the physician to view the internal anatomy of the patient. Over the years, numerous endoscopes have been developed and categorized according to specific applications, such as cystoscopy, colonoscopy, laparoscopy, upper GI endoscopy, and others. Endoscopes may be inserted into the body's natural orifices or through an incision in the skin. An endoscope is usually an elongated tubular shaft, rigid or flexible, having a video camera or a fiber optic lens assembly at its distal end. The shaft is connected to a handle, and viewing is usually possible via an external screen. Various surgical tools may be inserted through a working channel in the endoscope for performing different surgical procedures.

Endoscopic submucosal dissection (ESD) is an endoscopic procedure where physicians use an endoscope and other flexible endoscopic tools to remove cancerous and pre-cancerous lesions from a gastrointestinal tract. During such a procedure, an endoscope is often fitted with a clear distal end cap. This cap allows for improvement of optics or visualization during ESD by preventing red out, a circumstance where the user is unable to properly visualize the tissue due to the endoscopic camera being so close to the tissue. Distal end caps often have an inability to effectively drain fluid from the cap, which can create an impaired field of view of the endoscopic camera and increase the difficulty of using endoscopic accessory tools. If water, blood, mucus, or stool collects in a distal end cap, a user may have to remove the entire endoscope from a patient and clean the distal tip of the endoscope, increasing procedure time, procedure costs, and/or increasing the risk of injury to the patient.

The current disclosure may solve one or more of these issues or other issues in the art.

### SUMMARY

Embodiments of this disclosure relate to, among other things, medical devices and systems for visualizing internal patient anatomy, and related methods of use. Embodiments of this disclosure may provide space efficient component configurations within the tip section of a medical device, and/or may help to enable efficient packing of necessary elements in the tip section, while maintaining their functionality. Each of the embodiments disclosed herein may include one or more of the features described in connection with any of the other disclosed embodiments.

In some aspects, the techniques described herein relate to a cap for a medical device, including: a cylindrical body including a central lumen extending longitudinally through the cylindrical body, wherein a radially-inward facing surface of the cylindrical body extends around the central lumen; and a first protruding portion extending from the radially-inward facing surface, wherein the first protruding portion is configured to redirect fluid supplied from a fluid lumen of the medical device.

In some aspects, the cap may include one or more of the following features. The first protruding portion is triangular. The cap may further include a lateral lumen extending from a first opening at the radially-inward facing surface of the cylindrical body and a second opening at a radially-outward facing surface of the cylindrical body. The lateral lumen is longitudinally aligned with the first protruding portion. A second protruding portion may extend circumferentially around the radially-inward facing surface of the cylindrical body. The second protruding portion is configured to abut a distal face of the medical device. The second protruding portion is positioned at a longitudinal midpoint of the cylindrical body. The cylindrical body is configured to couple to a distal end of an endoscope. The first protruding portion includes a first planar surface transverse from a central longitudinal axis of the cylindrical body.

In some aspects, the cap may include one or more of the following features. The first protruding portion further includes a proximal end surface substantially parallel to a distal end surface of the cylindrical body. The first protruding portion includes an L-shaped cross-section and a first planar portion substantially perpendicular to a central longitudinal axis of the cylindrical body. The first protruding portion includes a living hinge structure. The first protruding portion is configured to rotate toward the radially-inward facing surface of the cylindrical body. The first protruding portion is curved and extends circumferentially around a central longitudinal axis of the cylindrical body. A second protruding portion extending circumferentially around the radially-inward facing surface of the cylindrical body; and a lateral lumen extending from the radially-inward facing surface to a radially-outward facing surface of the cylindrical body, wherein the lateral lumen is positioned distal from the first protruding portion and the second protruding portion; wherein the first protruding portion is configured to direct fluid from a fluid lumen of the medical device towards the lateral lumen.

In some aspects, the techniques described herein relate to a cap for a medical device, including: a cylindrical body including a central lumen extending longitudinally through the cylindrical body, wherein a radially-inward facing surface of the cylindrical body extends around the central lumen; and a first protruding portion extending from the radially-inward facing surface, wherein the first protruding portion is configured to redirect fluid supplied from a fluid lumen of the medical device.

In some aspects, the cap may include one or more of the following features. The first protruding portion is triangular. A lateral lumen may extend from a first opening at the radially-inward facing surface of the cylindrical body and a second opening at a radially-outward facing surface of the cylindrical body. The lateral lumen is longitudinally aligned with the first protruding portion. A second protruding portion may extend circumferentially around the radially-inward facing surface of the cylindrical body. The second protruding portion is configured to abut a distal face of the medical device. The second protruding portion is positioned at a longitudinal midpoint of the cylindrical body. The cylindrical body is configured to couple to a distal end of an endoscope. The first protruding portion includes a first planar surface transverse from a central longitudinal axis of the cylindrical body. The first protruding portion further includes a proximal end surface substantially parallel to a distal end surface of the cylindrical body. The first protruding portion includes an L-shaped cross-section and a first planar portion substantially perpendicular to a central longitudinal axis of the cylindrical body. The first protruding portion includes a living hinge structure. The first protruding portion is configured to rotate toward the radially-inward facing surface of the cylindrical body. The first protruding portion is curved and extends circumferentially around a central longitudinal axis of the cylindrical body. A second protruding portion extending circumferentially around the radially-inward facing surface of the cylindrical body; and a lateral lumen extending from the radially-inward facing surface to a radially-outward facing surface of the cylindrical body, wherein the lateral lumen is positioned distal from the first protruding portion and the second protruding portion; wherein the first protruding portion is configured to direct fluid from a fluid lumen of the medical device towards the lateral lumen.

In some aspects, the techniques described herein relate to a cap for a medical device, including: a cylindrical body including a central lumen extending longitudinally through the cylindrical body, wherein a radially-inward facing surface of the cylindrical body extends around the central lumen; and a first protruding portion extending from the radially-inward facing surface, wherein the first protruding portion extends circumferentially about a central longitudinal axis of the cylindrical body; wherein the first protruding portion includes a living hinge structure and is configured to rotate towards the radially-inward facing surface of the cylindrical body. In some aspects, the first protruding portion is configured to redirect fluid supplied from a fluid lumen of the medical device. In some aspects, the techniques described herein relate to a medical device, wherein the first protruding portion has an L-shaped cross-section. In some aspects, a lateral lumen extending from the radially-inward facing surface to a radially-outward facing surface of the cylindrical body.

In some aspects, the techniques described herein relate to a cap for a medical device, including: a cylindrical body including a central lumen extending longitudinally through the cylindrical body, wherein a radially-inward facing surface of the cylindrical body extends around the central lumen; a first protruding portion extending from the radially-inward facing surface, wherein the first protruding portion is triangular; a lateral lumen extending from the radially-inward facing surface to a radially-outward facing surface of the cylindrical body, wherein the lateral lumen is longitudinally aligned with the first protruding portion; and a second protruding portion extending circumferentially around the radially-inward facing surface, wherein the second protruding portion is configured to abut a distal face of the medical device; wherein the first protruding portion is configured to redirect fluid supplied from a fluid lumen of the medical device.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, device, or apparatus that comprises a list of elements does not include only those elements, but may include other elements not expressly listed or inherent to such process, method, article, device, or apparatus. The term “exemplary” is used in the sense of “example,” rather than “ideal.” The term “distal” refers to a portion farthest away from a user when introducing a device into a patient. By contrast, the term “proximal” refers to a portion closest to the user when placing the device into the patient. Proximal and distal directions are labeled with arrows marked “P” and “D”, respectively, throughout various figures. Although endoscopes are referenced herein, reference to endoscopes or endoscopy should not be construed as limiting the possible applications of the disclosed aspects. For example, the disclosed aspects may be used with duodenoscopes, bronchoscopes, ureteroscopes, colonoscopes, catheters, diagnostic or therapeutic tools or devices, or other types of medical devices. Further, relative terms such as, for example, “about,” “substantially,” “approximately,” etc., are used to indicate a possible variation of ±10% in a stated numeric value or range.

### BRIEF DESCRIPTION OF THE DRAWINGS

The accompanying drawings, which are incorporated in and constitute a part of this specification, illustrate exemplary aspects of this disclosure and together with the description, serve to explain the principles of the disclosure.

FIG. 1 is a perspective view of an endoscope system and a magnified view of the distal end of the endoscope of the endoscope system, according to aspects of this disclosure.

FIG. 2 is a perspective view of a distal tip section of a medical device, according to aspects of this disclosure.

FIG. 3 is a perspective view of a cap for use with a medical device, according to aspects of this disclosure.

FIG. 4 is a magnified, partial, cross-sectional view of the cap of FIG. 3, according to aspects of this disclosure.

FIG. 5 is a perspective view of another cap for use with a medical device, according to aspects of this disclosure.

### DETAILED DESCRIPTION

Reference will now be made in detail to aspects of this disclosure, examples of which are illustrated in the accompanying drawings. Wherever possible, the same or similar reference numbers will be used through the drawings to refer to the same or like parts.

Embodiments of this disclosure seek to improve caps for medical devices, and/or facilitate procedures involving a medical device with a cap. Embodiments of this disclosure seek to improve a physician's ability to clean a cap intraoperatively and/or operate a medical device, such as an endoscope, with a cap.

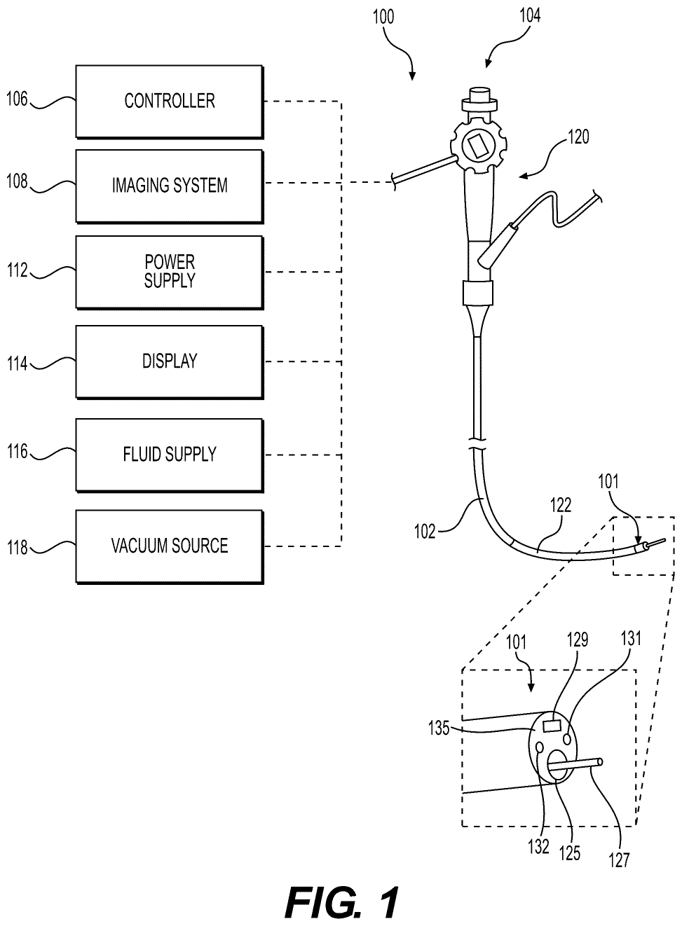

An exemplary endoscopy system 100 is shown in FIG. 1. Endoscopy system 100 may include an endoscope 104. Endoscope 104 may include a handle assembly 120 and a flexible tubular shaft 102. The flexibility of shaft 102 may be sufficient to allow shaft 102 to bend, to facilitate navigation of shaft 102 through a subject's tortuous anatomical passages. Shaft 102 may terminate at a distal tip 101. Shaft 102 may include an articulation section 122 for deflecting distal tip 101 in up, down, left, and/or right directions. In one example, articulation section 122 may provide for full retroflexion (e.g., rotation of distal tip 101 through an arc of 180 degrees) or only partial retroflexion (e.g., rotation of distal tip 101 through an arc of less than 180 degrees). Endoscope 104 also may include one or more lumens extending therethrough, and one or more openings in communication with the one or more lumens. For example, the one or more lumens may extend through handle assembly 120 and shaft 102, and the one or more openings may be on handle assembly 120 and distal tip 101. Endoscope 104 may be any suitable member for insertion into a patient's body, such as, e.g., an endoscope, a gastroscope, an ureteroscope, a nephroscope, a colonoscope, a hysteroscope, an ureteroscope, a bronchoscope, a cystoscope, a duodenoscope, a sheath, or a catheter.

One or more auxiliary devices may be operatively coupled to endoscope 104. Exemplary auxiliary devices may include a controller 106, an imaging system 108, a power supply 112, a display 114, a fluid supply 116, and/or a vacuum source 118, each of which is briefly described below. Controller 106 may include, for example, any electronic device capable of receiving, storing, processing, generating, and/or transmitting data according to instructions given by one or more programs. Controller 106 may be operatively coupled to, or part of, one or more of endoscope 104 and the other auxiliary devices, to control one or more aspects of their operation. Power supply 112 may include any suitable power source, and associated connectors (e.g., electrically-conductive wires), for supplying electronic components in the auxiliary devices and endoscope 104 with electrical power. Fluid supply 116 may include a reservoir, a medical irrigation bag, a pump, and any suitable connectors (e.g., tubing for fluidly coupling fluid supply 116 and endoscope 104). The pump may supply a flow of pressurized fluid to one or more of the lumens in endoscope 104, and the pressurized fluid flow may be emitted from distal tip 101 and/or used to inflate expandable components present at distal tip 101. Vacuum source 118 may provide suction or vacuum pressure to one or more lumens of the endoscope, and thereby provide a suction force or negative pressure to draw material toward and/or into endoscope 104, and/or to deflate expandable components.

Imaging system 108 may include imaging electronics to, for example, process signals received from an image sensor in endoscope 104, send signals for controlling the image sensor, adjust illumination levels of areas being viewed by the image sensor, and/or facilitate the display of image sensor data on display 114.

Distal tip 101 may include one or more image sensors 129 and one or more illuminators 131, shown in the magnified view of distal tip 101 in FIG. 1. One or more image sensors 129 may include a charge-coupled device image sensor, a complementary metal-oxide image semiconductor, or the like coupled to a cable or wire running through the shaft 102 of endoscope 104. One or more illuminators 131 may include light emitting diodes (LEDs) or the like. In some examples, one or more windows (not shown) may be in front of the one or more illuminators 131.

Distal tip 101 may include one or more fluid lumens 132. Fluid lumen 132 may extend from a proximal portion of endoscope 104 to distal tip 101, and may include an opening at distal front face 135 of distal tip 101. Fluid lumen 132 may be a fluid jet lumen and may include one or more nozzles at distal front face 135. Fluid lumen 132 may be configured to jet fluid away from distal front face 135. For example, fluid lumen 132 may be configured to jet fluid away from distal front face 135 in a direction perpendicular to distal front face 135. In other examples, fluid lumen 132 may be configured to jet fluid away from distal front face 135 in a direction that forms an acute angle with distal front face 135. In some examples, fluid lumen 132 may be configured to jet fluid substantially tangentially and/or across distal front face 135, for example to clean image sensors 129 or a window on distal front face 135 to image sensors 129. The embodiments of distal end caps discussed herein below may be configured to leverage one or more fluid lumens 132 to clean the distal end cap.

A tool 127 may be inserted into a working channel or lumen 125 of endoscope 104, and tool 127 may exit out of the distal end of lumen 125. Tool 127 may include, for example, a brush, such as a wire brush, a guidewire, cutting or grasping forceps, a biopsy device, a snare loop, an injection needle, a cutting blade, an electrosurgical knife, scissors, a retractable basket, a retrieval device, an ablation and/or electrophysiology catheter, a stent placement device, a surgical stapling device, a balloon catheter, a laser-emitting device, and/or any other suitable therapeutic or diagnostic accessory device. As shown in the magnified view of distal tip 101, tool 127 has a smaller circumference about its longitudinal axis compared to the circumference about the longitudinal axis of lumen 125, and may include a smaller cross-sectional diameter as compared to the diameter of lumen 125.

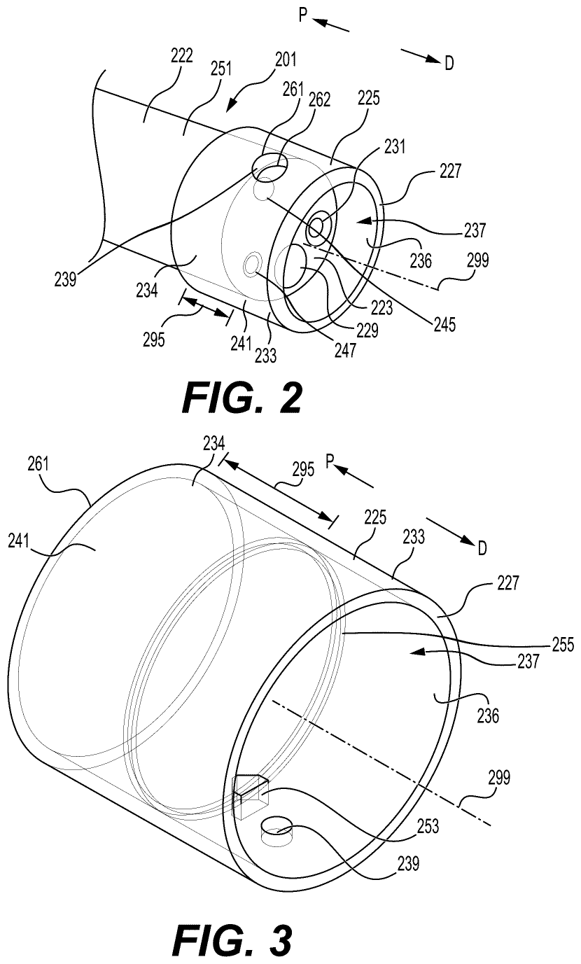

FIG. 2 illustrates an exemplary distal portion 201 of a medical device, such as endoscope 104, with a cap 225 positioned over distal portion 201. Cap 225 is shown as transparent to show a distal end face 223 of distal portion 201. Distal portion 201 may include a camera or other image sensor 231, a working channel 229, an illuminator 247, and a fluid lumen 245, such as a fluid jet lumen. Cap 225 may be cylindrical and may include a central lumen 237 formed by a curved, radially-inward facing surface 236, relative to a central longitudinal axis 299 of cap 225. Central lumen 237 may be sized to receive a shaft 222 of a medical device, such that cap 225 can be removably coupled to shaft 222. Central lumen 237 may have a diameter that is substantially equal to the diameter of shaft 222. Radially-inward facing surface 236 of cap 225 may be circular and may be configured to abut with a radially-outward facing surface 251 of shaft 222, and radially-inward facing surface 236 may extend perpendicular from distal end face 223. Cap 225 may be configured to fit over the distal end face 223 of shaft 222, such that a first portion 233 of cap 225 extends distally from distal end face 223 and a second portion 234 (shown by a length identifier arrow 295 across the second portion 234 of cap 225) extends proximally from distal end face 223 when cap 225 is coupled to shaft 222. In some examples, first portion 233 of cap 225 may be half of cap 225, and second portion 234 of cap 225 may be the other half of cap 225. In other examples, first portion 233 of cap 225 may be a third of cap 225, and second portion 234 may be two thirds of cap 225. In other examples, first portion 233 may be any other portion of cap 225 besides half or a third, and second portion may be the remaining portion of cap 225. Length identifier arrow 295 also designates the overlapping portion of cap 225 with shaft 222. A distal front face 227 of cap 225 may be entirely distal from distal end face 223 of shaft 222, and may be circular.

In some examples, distal front face 227 may be curved and may be configured to be atraumatic when abutting tissue of a patient. In some examples, lumen 237 may have a smaller diameter across the first portion 233 of cap 225, and larger diameter across the second portion 234 of cap to provide a stop ledge extending circumferentially around radially-inward facing surface 236, which may abut a portion (e.g., a radially outer portion) of distal end face 223 when cap 225 is coupled to shaft 222.

A lateral lumen 239 may extend through first portion 233 of cap 225, and may include a first opening 261 at a radially-outermost surface 241, relative to central longitudinal axis 299, and a second opening 262 at radially-inward facing surface 236. Lateral lumen 239 may be configured to be a fluid exit lumen and to allow fluid to flow from an interior portion of cap 225 (e.g., within lumen 237) to outside of cap 225. Lateral lumen 239 may be positioned in first portion 233 of cap 225 such that, when cap 225 is coupled to shaft 222, lateral lumen 239 is entirely distal from distal end face 223. In other examples (not shown), lateral lumen 239 may be partially distal of distal end face 223 when cap 225 is coupled to shaft 222. As shown in FIG. 3, lateral lumen 239 may be longitudinally aligned with and positioned distal from a protruding portion 253 of cap.

FIG. 3 illustrates a perspective view of cap 225, with cap 225 shown transparent to expose features of cap 225. A protrusion 255 may extend around the circumference of radially-inward facing surface 236, and may protrude towards central longitudinal axis 299 of cap 225. Although shown circular, protrusion 255 may be any suitable shape, may only extend across a portion of the circumference of radially-inward facing surface 236, may include a plurality of protrusions, and/or may be rectangular or any other shape. Protrusion 255 may be positioned at a longitudinal mid-point of cap 225, or may be positioned at any other position along the length of cap 225. Protrusion 255 may be configured to abut distal end face 223, and may help prevent a user from pushing cap 225 further onto shaft 222. In these aspects, protrusion 255 may help to allow a user to properly position cap 225 onto a shaft 222 of a medical device.

Protruding portion 253 may be positioned distal from protrusion 255, and may be proximate to lateral lumen 239. In other examples, protruding portion 253 may be positioned on an opposite side of central lumen 237 from lateral lumen 239, or may be positioned at any other position distal from protrusion 255. Protruding portion 253 may be triangular and may be configured to redirect fluid moving out of a fluid lumen 245 of a medical device. A corner of the triangular shape of protruding portion 253 may be pointed in the proximal direction and may be configured to align with a fluid lumen 245 of a medical device. Protruding portion 253 may be configured to help redirect fluid movement towards radially-inward facing surface 236, which may help facilitate cleaning debris from cap 225.

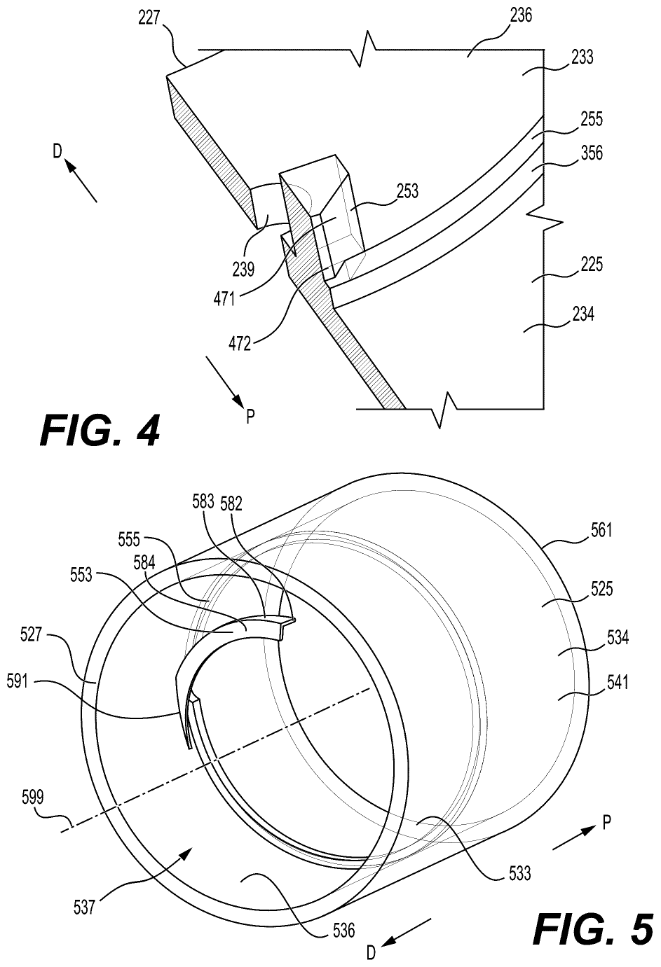

FIG. 4 illustrates a magnified portion of cap 225, with a portion shown in cross-hatch. Protruding portion 253 may be configured to be positioned within the path of fluid movement from fluid lumen 245. A lateral surface 471 of protruding portion 253 may be planar and be angled relative to central longitudinal axis 299 of cap 225. Lateral surface 471 may be configured to redirect fluid movement from fluid lumen 245. As shown in FIG. 4, protruding portion 253 may abut protrusion 255. In other examples, protruding portion 253 may be spaced from protrusion 255. A proximal most surface 472 of protruding portion 253 may be substantially perpendicular to central longitudinal axis 299. Surfaces 471, 472 may be configured to divert fluid flow from fluid lumen 245 towards radially-inward facing surface 236 of cap 225, to clean cap 225. In some examples, protruding portion 253 may be configured to broadcast or otherwise direct fluid from fluid lumen 245 in a plurality of directions, such as in a circular cone shape or a fan shape pattern. The redirected fluid from fluid lumen 245, after deflecting off of protruding portion 253, may be configured to direct debris directly out of cap 225 and/or towards lateral lumen 239. A proximal-facing surface 356 of protrusion 255 may be configured to abut distal end face 223.

In operation, a user may couple cap 225 to shaft 222 by sliding second portion 234 of cap 225 onto shaft 222 (e.g., proximally) until protrusion 255 abuts distal end face 223. In some examples, cap 225 may not include protrusion 255 and a user may simply slide cap 225 over shaft 222. The user may then, in some examples, align protruding portion 253 longitudinally with fluid lumen 245. The user may then actuate fluid supply, for example, via controller 106, to supply fluid lumen 245 with fluid flow. Once the fluid flow is activated, the fluid may expel out of fluid lumen 245, deflect from protruding portion 253, and move towards radially-inward facing surface 236 of cap 225 to help clean cap 225. In some examples, the fluid flow may move debris out of central lumen 237 and/or out of lateral lumen 239.

FIG. 5 illustrates a perspective view of an alternative embodiment of cap 525. Cap 525 is shown as transparent to facilitate viewing portions of cap 525, and cap 525 may have any of the features described herein with relation to cap 225. Cap 525 may include a proximal end 561, a distal end face 527, a first portion 533, a second portion 534, a radially-outward facing surface 541, a radially-inward facing surface 536, a protruding portion 553, a circular protruding portion 555, and a central lumen 537. Circular protruding portion 555 may be substantially the same as protrusion 255.

As shown in FIG. 5, protruding portion 553 may have a curved body extending longitudinally from radially-inward facing surface 536 toward central longitudinal axis 599 of cap 525. Protruding portion 553 may have an L-shaped cross-section, with a first planar portion 583 and a second planar portion 584. First planar portion 583 may be substantially parallel to central longitudinal axis 599, and second planar portion 584 may be substantially perpendicular to central longitudinal axis 599. Protruding portion 553 may extend across the distal opening of fluid lumen 245 (FIG. 2), and may be configured to divert fluid flow towards radially-inward facing surface 536 of cap 525. Protruding portion 553 may be configured to not block a working channel 125, 229 of the medical device. In some examples, protruding portion 553 may be curved and may extend around central longitudinal axis 599. Protruding portion 553 may extend radially-inward from radially-inward facing surface 536 to a first end 582 of protruding portion 553.

Protruding portion 553 may be moveable, and may be a living hinge structure. For example, protruding portion 553 may include a living hinge structure that is configured to allow protruding portion 553 to rotate about a pivot point 591. Although not shown, in some examples protruding portion 553 may be rotatable to a position substantially flush with radially-inward facing surface 536. Protruding portion 553 may be biased towards an extended position, as shown in FIG. 5, and may be rotatable to a position substantially flush with radially-inward facing surface 536 when a threshold amount of force is applied to protruding portion 553.

In some examples, protruding portion 553 may be configured to direct fluid towards radially-inward facing surface 536 when a user provides a small amount of water through fluid lumen 245 (e.g., a short burst of fluid ejected from fluid lumen 245, such as an application of fluid flow to fluid lumen 245 for 3 or less seconds). In such an example, when a user maintains the application of fluid flow through fluid lumen 245 for an extended period of time (e.g., more than 3 seconds or any other threshold time period, such as more than 5, 10, 15, or 30 seconds), protruding portion 553 may rotate, via the force applied by the fluid flow from fluid lumen 245, to a retracted position in which protruding portion 553 is substantially flush with radially-inward facing surface 536. When protruding portion 553 is rotated to a retracted position, fluid lumen 245 may operate normal irrigation, for example, in which fluid is expelled distally towards a body lumen of a patient.

Any of the caps 225, 525 described herein may be made of any suitable material, such as plastic, PEBAX, or any other suitable material. Any of the caps 225, 525 may be unitary or made of a plurality of materials. In some examples, caps 225 may be made of any suitable polymer material, and may be optically transparent or partially transparent. In any of the above-described embodiments, caps 225, 525 may include one or more components that are metallic, a polymer, machined, formed, stamped, insert molded, or any combination thereof. Any aspect(s) of any one of the above-described embodiments of caps 225, 525 may be incorporated into any of the other caps 225, 525 described herein.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A cap for a medical device, comprising:
a cylindrical body including a central lumen extending longitudinally through the cylindrical body, wherein a radially-inward facing surface of the cylindrical body extends around the central lumen; and
a first protruding portion extending from the radially-inward facing surface, wherein the first protruding portion is configured to redirect fluid supplied from a fluid lumen of the medical device.

2. The cap of claim 1, wherein the first protruding portion is triangular.

3. The cap of claim 1, further comprising a lateral lumen extending from a first opening at the radially-inward facing surface of the cylindrical body and a second opening at a radially-outward facing surface of the cylindrical body.

4. The cap of claim 3, wherein the lateral lumen is longitudinally aligned with the first protruding portion.

5. The cap of claim 1, further comprising a second protruding portion extending circumferentially around the radially-inward facing surface of the cylindrical body.

6. The cap of claim 5, wherein the second protruding portion is configured to abut a distal face of the medical device.

7. The cap of claim 6, wherein the second protruding portion is positioned at a longitudinal midpoint of the cylindrical body.

8. The cap of claim 1, wherein the cylindrical body is configured to couple to a distal end of an endoscope.

9. The cap of claim 1, wherein the first protruding portion includes a first planar surface transverse from a central longitudinal axis of the cylindrical body.

10. The cap of claim 9, wherein the first protruding portion further includes a proximal end surface substantially parallel to a distal end surface of the cylindrical body.

11. The cap of claim 1, wherein the first protruding portion includes an L-shaped cross-section and a first planar portion substantially perpendicular to a central longitudinal axis of the cylindrical body.

12. The cap of claim 11, wherein the first protruding portion includes a living hinge structure.

13. The cap of claim 12, wherein the first protruding portion is configured to rotate toward the radially-inward facing surface of the cylindrical body.

14. The cap of claim 1, wherein the first protruding portion is curved and extends circumferentially around a central longitudinal axis of the cylindrical body.

15. The cap of claim 13, further comprising:
a second protruding portion extending circumferentially around the radially-inward facing surface of the cylindrical body; and
a lateral lumen extending from the radially-inward facing surface to a radially-outward facing surface of the cylindrical body, wherein the lateral lumen is positioned distal from the first protruding portion and the second protruding portion;
wherein the first protruding portion is configured to direct fluid from a fluid lumen of the medical device towards the lateral lumen.

16. A cap for a medical device, comprising:
a cylindrical body including a central lumen extending longitudinally through the cylindrical body, wherein a radially-inward facing surface of the cylindrical body extends around the central lumen; and
a first protruding portion extending from the radially-inward facing surface, wherein the first protruding portion extends circumferentially about a central longitudinal axis of the cylindrical body;
wherein the first protruding portion includes a living hinge structure and is configured to rotate towards the radially-inward facing surface of the cylindrical body.

17. The cap of claim 16, wherein the first protruding portion is configured to redirect fluid supplied from a fluid lumen of the medical device.

18. The medical device of claim 17, wherein the first protruding portion has an L-shaped cross-section.

19. The medical device of claim 18, further comprising a lateral lumen extending from the radially-inward facing surface to a radially-outward facing surface of the cylindrical body.

20. A cap for a medical device, comprising:
a cylindrical body including a central lumen extending longitudinally through the cylindrical body, wherein a radially-inward facing surface of the cylindrical body extends around the central lumen;
a first protruding portion extending from the radially-inward facing surface, wherein the first protruding portion is triangular;
a lateral lumen extending from the radially-inward facing surface to a radially-outward facing surface of the cylindrical body, wherein the lateral lumen is longitudinally aligned with the first protruding portion; and
a second protruding portion extending circumferentially around the radially-inward facing surface, wherein the second protruding portion is configured to abut a distal face of the medical device;
wherein the first protruding portion is configured to redirect fluid supplied from a fluid lumen of the medical device.
